(19) United States
(12) Patent Application Publication
(10) Pub. No.: US 2016/0038609 A1
(43) Pub. Date: Feb. 11, 2016

(54) Multistage Nanoparticles

(71) Applicants: The General Hospital Corporation, Boston, MA (US); Massachusetts Institute of Technology, Cambridge, MA (US)

(72) Inventors: Cliff Wong, Tucson, AZ (US); Moungi G. Bawendi, Cambridge, MA (US); Triantafyllos Stylianopoulos, Nicosia (CY); Rakesh K. Jain, Wellesley, MA (US); Dai Fukumura, Newton, MA (US)

(21) Appl. No.: 14/642,455

(22) Filed: Mar. 9, 2015

Related U.S. Application Data
(63) Continuation of application No. 13/821,166, filed on May 10, 2013, now abandoned.
(60) Provisional application No. 61/385,054, filed on Sep. 21, 2010.

(51) Int. Cl. A61K 47/48; A61K 45/06

(52) U.S. Cl. CPC A61K 47/48884; A61K 45/06

(57) ABSTRACT

Multistage nanostructures, e.g., for delivery of agents such as imaging agents and therapeutic agents to tumor vasculature.

### PRIORITY CLAIM

This application claims the benefit of U.S. Provisional Patent Application No. 61/385,054, filed Sep. 21, 2010. The entire contents of the foregoing are incorporated herein by reference.

### FEDERALLY SPONSORED RESEARCH OR DEVELOPMENT

This invention was made with Government support under Grant Nos. R01-CA126642, R01-CA085140, R01-CA115767, P01-CA080124, 1U54-CA119349, and R01-CA096915 awarded by the National Institutes of Health, and W911NF-07-D-0004 awarded by the U.S. Army. The Government has certain rights in the invention.

### TECHNICAL FIELD

This invention relates to multistage nanostructures, e.g., for delivery of agents such as imaging agents and therapeutic agents, to tumor vasculature.

### BACKGROUND

Nanoparticles (NPs) have offered new approaches to the delivery of cancer therapeutics (Jain R K & Stylianopoulos T (2010), Nature Reviews Clinical Oncology AOP; Schroeder A, Levins C G, Cortez C, Langer R, & Anderson D G (2010), Journal of Internal Medicine 267(1):9-21; Farokhzad O C, et al. (2006), Proceedings of the National Academy of Sciences 103(16):6315-6320; Duncan R (2006), Nature Reviews Cancer 6(9):688-701; Davis M E, et al. (2010), Nature 464(7291):1067-1070). Doxil® (˜100 nm PEGylated liposomal form of doxorubicin) and Abraxane® (˜130 nm albumin bound paclitaxel nanoparticle) are two examples of FDA-approved nanoparticle-based therapeutics for solid tumors; their large size compared to conventional cancer therapeutics allows them to preferentially accumulate in solid tumors by the EPR effect (Perrault S D, Walkey C, Jennings T, Fischer H C, & Chan W C W (2009), Nano Letters 9(5):1909-1915), thus reducing normal tissue toxicity. However, despite the improved pharmacokinetic properties (Soo Choi H, et al. (2007), Nat Biotech 25(10):1165-1170) and reduced adverse effects, these drugs have provided only modest survival benefits (Jain R K & Stylianopoulos T (2010), Nature Reviews Clinical Oncology AOP; Winer E P, et al. (2004), Journal of Clinical Oncology 22(11):2061-2068; Gradishar W J, et al. (2005), J Clin Oncol 23(31):7794-7803; O'Brien M E R, et al. (2004), Annals of Oncology: Official Journal of the European Society for Medical Oncology/ESMO 15(3):440-449). This is likely attributed to the physiological barriers imposed by the abnormal tumor vasculature and the dense interstitial matrix—a complex assembly of collagen, glycosaminoglycans, and proteoglycans—which hinder delivery of the drug throughout the entire tumor in sufficient concentration (Jain R K (1998), Journal of Controlled Release 53(1-3):49-67; Jain R K (2008), Scientific American 298(426):56-63).

Systemic delivery of therapeutics to the tumor is a three step process: blood-borne delivery to different regions of the tumor, transport across the vessel wall, and passage through the interstitial space to reach the tumor cells (Jain R K (1999), Annual Review of Biomedical Engineering 1(1):241-263). Abnormalities in the tumor vasculature lead to highly heterogeneous vascular perfusion throughout the tumor. The microvascular density is high at the invasive edge of the tumor but sometimes the tumor center is unperfused, preventing delivery of therapeutics to this region. However, the latter region's hostile microenvironment (low pH and low pO₂) harbors the most aggressive tumor cells and the tumor will regenerate if these cells are not eliminated. Moreover, exposure of the cancer cells to sublethal concentration of the therapeutic agent may facilitate the development of resistance.

Hyper-permeability of the abnormal vasculature and lack of functional lymphatics lead to elevated levels of interstitial fluid pressure (IFP) (Jain R K & Baxter L T (1988), Cancer Research 48(24 Part 1):7022-7032; Boucher Y, Baxter L T, & Jain R K (1990), Cancer Research 50(15):4478-4484). This interstitial hypertension in turn reduces convective transport across the vessel wall and into the interstitial space, leaving diffusion as the primary mode for drug transport to the poorly perfused regions. Large 100-nm nanostructure s are suitable for the EPR effect but have poor diffusion in the dense collagen matrix of the interstitial space (McKee T D, et al. (2006), Cancer Res 66(5):2509-2513; Netti P A, Berk D A, Swartz M A, Grodzinsky A J, & Jain R K (2000), Cancer Research 60(9):2497-2503), resulting in restrictive nanoparticle accumulation around tumor blood vessels and little penetration into the tumor parenchyma. In the case of Doxil® for example, the liposomal particles are trapped close to the vasculature. Although the small size (˜400 MW) of the doxorubicin, that is released from the liposomes, seemingly allows rapid diffusion, it cannot migrate far from the particles due to avid binding to DNA and sequestration in acidic endosomes of tumor cells (Ouar Z, et al. (2003), Biochemical Journal 370(Pt 1):185-193; Primeau A J, Rendon A, Hedley D, Lilge L, & Tannock I F (2005), Clinical Cancer Research 11(24):8782-8788), resulting in heterogeneous therapeutic effects.

### SUMMARY

Described herein is a multistage system in which a nanostructure (e.g., of about 100 nm) “shrinks” its size to a nanoparticle (e.g., of about 10 nm) after it extravasates from leaky regions of the tumor vasculature and is exposed to tumor microenvironment. The shrunken nanoparticles can more readily diffuse throughout the tumor's interstitial space. This size change is triggered by proteases that are highly expressed in the tumor microenvironment such as MMP-2 and MMP-9 that degrade the cores of gelatin nanostructures, e.g., of about 100-nm in diameter, and release smaller nanoparticles, e.g., of about 10-nm in diameter, from its surface.

Thus, in one aspect the present invention features compositions including a nanostructure comprising gelatin at least one nanoparticle incorporated therein (the nanoparticle is also referred to herein as an inner core nanoparticle, though it need not be on the “inside” of the nanostructure, so long as the gelatin is accessible to the action of a matrix metalloprotease (MMP) to allow for digestion of the gelatin to release the nanoparticle and reduce the size of the composition). The nanoparticle can include one or both of a cancer therapeutic agent or an imaging agent. The gelatin nanostructure is subject to degradation (i.e., can be degraded) by a MMP, e.g., MMP-2 and/or MMP-9. The degradation releases the nanoparticle (which is not sensitive to degradation by MMPs).

In some embodiments, the nanoparticle is includes one or more members of the group consisting of quantum dots, polymers, liposomes, silicon, silica, dendrimers, microbubbles, and nanoshells. In some embodiments, the nanoparticle comprises one or more quantum dots, e.g., CdSe quantum dots.

In some embodiments, the gelatin nanostructure has a charged outer surface, e.g., comprised of a material selected from the group consisting of polyethylene glycol (PEG), N-(2-hydroxypropyl)methacrylamide (HPMA), poly(vinyl-pyrrolidone) (PVP), poly(ethyleneimine) (PEI), a polyamidoamine, a mixture of divinyl ether and maleic anhydride (DIVEMA (DIVEMA), dextran (alpha-1,6 polyglucose, dextrin (alpha-1,4 polyglucose), hyaluronic acid, a chitosan, a polyamino acid, poly(lysine), poly(glutamic acid), poly(malic acid), poly(sapartamides), poly co-polymers, and copaxone.

In some embodiments, the nanostructure is about 10-300 nm in diameter, e.g., about 100 nm in diameter. In some embodiments, the nanoparticle is about 2-20 nm in diameter, e.g., about 10 nm in diameter.

In another aspect, the present invention provides methods for delivering a cancer therapeutic or an imaging agent to a tumor in a subject. The methods include administering to the subject an effective amount of a nanostructure as described herein.

In a further aspect, the present invention features methods for treating cancer in a subject. The methods include administering to the subject a therapeutically effective amount of a nanostructure composition as described herein, thereby treating the subject.

In yet another aspect, the present invention provides methods for inhibiting the growth of a tumor in a subject, the method comprising administering to the subject a therapeutically effective amount of a composition as described herein, thereby inhibiting the growth of the tumor in the subject.

In an additional aspect, the invention features methods for delivering an imaging agent to a tumor, the method comprising administering to a subject a composition as described herein that includes an imaging agent to the subject.

Also provided herein are pharmaceutical compositions including the nanostructure compounds described herein and a pharmaceutically acceptable carrier.

Further, described herein is the use of the compounds and compositions described herein to treat or image cancer in a subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. QDGelNPs changes it size in response to MMP-2. 1(A) Schematic of 100 nm QDGelNPs changing size to 10 nm QD NPs by cleaving away the gelatin scaffold with MMP-2, a protease highly expressed in tumor tissue. (1B) GFC chromatograms of QDGelNPs at various times after incubation with MMP-2. Fluorescence signal at 565 nm is collected. (Inset) Fluorescence spectrum of the peak at void volume for 2.2 hours cleaving time shows signal originates from QDs on the QDGelNPs. (1C) Epifluorescence image of QDGelNPs on a silicon substrate at 100× magnification. Scale bar 5 μm (1D) SEM image of QDGelNPs at 15,000× magnification. Scale bar 1 μm. (Inset, bottom) SEM image of individual QDGelNPs at 35,000× magnification. Scale bar, 100 nm.

FIG. 1E. Histogram of QDGelNPs size distribution from image analysis of SEM micrograph.

FIG. 1F. DLS distribution of QDGelNP on day 1 and day 48 after synthesis and storage at 4° C.

FIG. 1G. GFC chromatograms of QDGelNPs and QDGelNPs incubated with 50% FBS for 13 hours indicate no peak corresponding to release of individual QDs. Chromatograms of QDs and QDs incubated with 95% FBS are provided as reference.

FIGS. 2A-B. Kinetics of MMP-2 induced QD release from QDGelNPs. (2A) QD-release curve from incubation of 0.1 mg of QDGelNPs with 235 ng of MMP-2 (2B) QD release from incubation of 0.1 mg of QDGelNPs for 12 hours with varying amounts of MMP-2.

FIGS. 3A-B. FSC cross-correlograms of QDGelNPs before (3A) and after (3B) incubation with MMP-2.

FIGS. 4A-H. Diffusion of SilicaQDs and QDGelNPs (before and after MMP-2 cleavage) in a collagen gel. (4A, 4B) Fluorescence images of SilicaQDs (4A) and QDGelNPs before MMP-2 cleavage (4B) penetrating into the collagen gel. (4C) Second-harmonic generation (SHG) signal shows the corresponding location of the collagen matrix. Scale bars, 125 μm. (4D) Normalized intensity profile of SilicaQDs and QDGelNPs in collagen gel. (4E, 4F) Fluorescence images of SilicaQDs (4E) and QDGelNPs after MMP-2 cleavage (4F) penetrating into the collagen gel. (4G) SHG signal shows the corresponding location of the collagen matrix. Scale bars 125 (4H) Normalized intensity profile of SilicaQDs and QDGelNPs after MMP-2 cleavage in collagen gel. Black line displays theoretical intensity profile for particles with diffusion coefficient of 2.3×10⁻⁷ cm² s⁻¹.

FIGS. 4I-J. Particle distribution and second-harmonic generation (SHG) from collagen fibrils at collagen-solution interface in collagen gel experiment (FIGS. 4A-H). (4I) The intensity profile of SilicaQDs and QDGelNPs before cleavage in comparison with SHG indicates the exclusion of both sets of particles from the collagen matrix. (4J) Intensity profile of SilicaQDs and QDGelNPs after cleavage in comparison with SHG shows penetration of QDGelNPs into the collagen but exclusion of SilicaQDs.

FIGS. 5A-F. In vivo images of QDGelNPs and SilicaQDs after intratumoral co-injection into HT-1080 tumor. QDGelNPs imaged 1 hour (5A), 3 hours 5B), and 6 hours (5C) post-injection. SilicaQDs imaged 1 hour (5D), 3 hours (5E), and 6 hours (5F) injection. Scale bar 100 μm.

FIG. 6. An exemplary schematic depiction of one embodiment of the multistage nanoparticle drug delivery system. The initial 100-nm multistage nanostructure delivery system accumulates preferentially around leaky vessels in tumor tissue. Due to its large size, the 100-nm nanostructure cannot penetrate the dense collagen matrix of the interstitial space. However, endogenous MMPs can proteolytically degrade the gelatin core of the 100-nm nanostructure, releasing smaller 10-nm nanoparticles from its surface, which can penetrate deep into the tumor due to their small size and PEGylated (“stealthy”) surface. After disseminating all through the tumor, the 10-nm nanoparticles can serve as depots for drugs that are released uniformly throughout the tumor.

FIGS. 7A-D. DLS size and zeta potential distributions of QDGelNPs and QDs. DLS mass percent particle size distributions of QDGelNPs (7A) and QD (7B). Zeta Potential distributions of QDGelNPs (7C) and QDs (7D).

FIG. 8. Blood concentration of QDGelNPs and SilicaQDs as a function of time post-injection. Error bars indicate S.E.M. of three animals.

### DETAILED DESCRIPTION

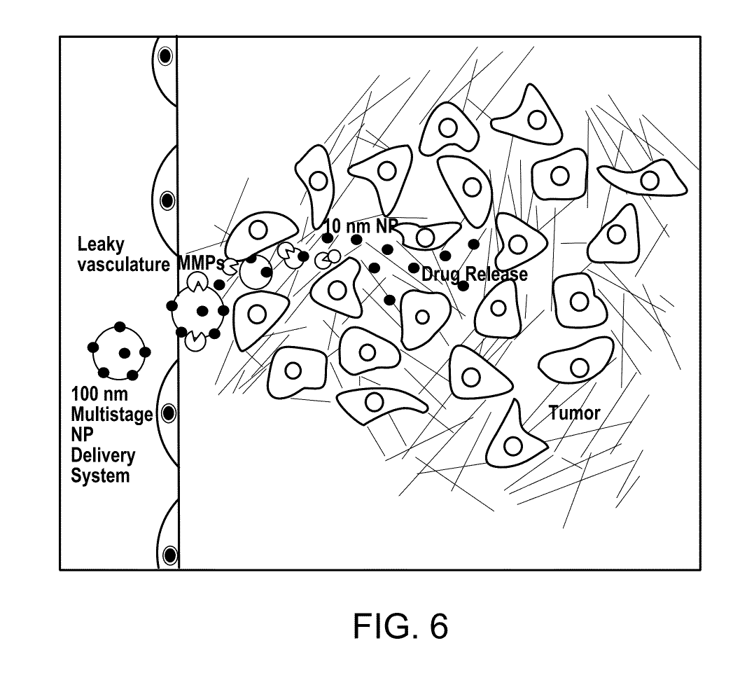

The present invention is based, at least in part, on the development of a multistage system in which nanoparticles change their size to facilitate transport by adapting to each physiological barrier. The original nanostructures preferentially extravasate from the leaky regions of the tumor vasculature. After extravasation into tumor tissue, the nanostructure “shrink,” e.g., to the size of the core nanoparticles (in some embodiments, about 10 nm), significantly lowering their diffusional hindrance in the interstitial matrix (Pluen A, et al. (2001), Proceedings of the National Academy of Sciences of the United States of America 98(8):4628-4633) and allowing penetration into the tumor parenchyma. These smaller nanoparticles can potentially be used as nanocarriers for therapeutics that are released as the particles penetrate deep into the tumor (FIG. 6). Surface PEGylation of the small nanoparticles allows it to diffuse smoothly in the interstitial matrix by reducing the binding, sequestration, and metabolism, which hinder the transport of much smaller therapeutic agents (Jain R K (1987), Cancer Research 47(12):3039-3051; Minchinton A I & Tannock I F (2006), Nat Rev Cancer 6(8):583-592). Furthermore, the core nanoparticles are not cleared from the tumor as rapidly as much smaller molecular species due to their larger size.

To achieve this size shrinking property, a large nanostructure should be triggered to release smaller nanoparticles after extravasation into the tumor. Several nanostructure have been designed to release their contents remotely via an external stimulus (light, heat, ultrasound, magnetic field, etc.), but their use to date has been limited to local therapy. Systemic therapy is necessary to treat the metastases, which are the major cause of cancer mortality. Water hydrolysis—(Gref R, et al. (1994), Science 263(5153):1600-1603), diffusion-, or solvent-controlled release mechanisms can achieve systemic effects but do not give preferential release in the tumor, resulting in increased toxicity in normal tissues. To attain both systemic therapeutic effects and preferential release in tumor tissues, in the present methods and compositions the size change is triggered using an endogenous stimulus characteristic of the tumor microenvironment, such as low pH, low partial oxygen pressure, or high concentrations of matrix metalloproteinases (MMPs). Acidic and hypoxic regions tend to be far from blood vessels (Helmlinger G, Yuan F, Dellian M, & Jain R K (1997), Nat Med 3(2):177-182), not in the perivascular regions where the large nanoparticles are trapped. MMPs, particularly gelatinases A and B (MMP-2 and -9), are key effectors of angiogenesis, invasion, and metastasis including the epithelial-mesenchymal transition (EMT), a cell-biological program which executes many of the steps of the invasion-metastasis cascade. They cleave away the extracellular matrix (ECM); creating space for the cell to move and releasing sequestered growth factors (Roy R. Zhang B. Moses M A (2006), Experimental Cell Research. 312(5):608-622; Deryugina E I, Bourdon M A, Reisfeld R A, & Strongin A (1998), Cancer Res 58(16):3743-3750). Levels of MMP-2 and -9 are high at the invasive edge of tumors and at the sites of angiogenesis—regions the large nanoparticles are likely to extravasate. These conditions make enzymatic degradation by MMPs a highly favorable trigger mechanism.

Because both MMP-2 and -9 are extremely efficient at hydrolyzing gelatin (denatured collagen), a nanostructure of about 100-nm was engineered with a core composed of gelatin and a surface covered with quantum dots (QDs), a model nanoparticle of about 10-nm. Gelatin nanoparticles have been shown to have a long blood circulation time and high accumulation in tumor tissues (Kaul G & Amiji M (2004), Journal of Drug Targeting 12(9):585), properties necessary for the first-stage NP carrier. In order to access the spatial and temporal distribution of the nanoparticles in the tumor milieu, ˜10 nm QDs were used as a stand-in for therapeutic nanocarriers, so that the nanostructures/nanoparticles' distribution in vivo can be imaged using time-lapse multiphoton microscopy. Compared to traditional organic fluorophores, QDs have high resistance to photo- and chemical-degradation, narrow photoluminescence spectrum, broad excitation spectral window, and large two-photon absorption cross-section—enabling the use of multiphoton microscopy to image deep into the tumor with high spatial resolution (Stroh M, et al. (2005), Nat Med 11(6):678-682).

In some embodiments, there are three main criteria for the multistage QDGelNPs: the nanostructure/nanoparticles' size must change, e.g., from 100 nm to 10 nm, their surface before and after MMP-2 cleavage should to be well PEGylated and preferably neutral, and their sensitivity to cleavage should be at least as high as other reported MMP-2 probes (Chau Y, Tan F E, & Langer R (2004), Bioconjugate Chemistry 15(4):931-941; Harris T J, Maltzahn Gv, Derfus A M, Ruoslahti E, & Bhatia S N (2006), Angewandte Chemie International Edition 45(19):3161-3165; Bremer C, Tung C-H, & Weissleder R (2001), Nat Med 7(6):743-748). Satisfying these three criteria simultaneously presented several design and synthetic challenges. However, optimizing the coupling scheme and the degree of glutaraldehyde cross-linking met the desired criteria for this system while preserving the simplicity in design to ensure scalability.

Since in some embodiments both the nanostructure carrier and released nanoparticles should be highly PEGylated and neutral or with a specific charge, encapsulation strategies that rely on hydrophobic or charged interactions are unsuitable. Using QDs with a “sticky” surface (low coverage of PEG) allowed for encapsulation inside the gelatin core, but the particle after cleavage showed broad size distribution by GFC, indicating binding to gelatin/glutaraldehyde fragments. An alternative strategy is to covalently attach the QDs onto the gelatin nanostructure surface; however, this approach is susceptible to interparticle cross-linking. By careful optimization and utilizing EDC/sulfo-NHS chemistry to conjugate amino-PEG QDs to the carboxylic acid groups on the gelatin nanostructure surface, QDGelNPs without significant interparticle cross-linking were produced and the cleaved QDs' size increase.

By controlling the length and degree of glutaraldehyde polymerization used to cross-link the gelatin nanoparticles, the size of the QDs after cleaving and their rate of release while maintaining particle stability can be optimized. The method for gelatin nanoparticle synthesis developed in (Coester C J, Langer K, Von Briesen H, & Kreuter J (2000), Journal of Microencapsulation: Micro and Nano Carriers 17(2):187) produced gelatin nanostructures with long extended networks of glutaraldehyde on their surface to maintain particle stability in aqueous solution. However, when the same scheme was applied to this design, the QDs remain covalently attached to this large glutaraldehyde polymer after MMP-2 degradation, resulting in QDs that were significantly larger upon release. To produce released QDs without augmenting their size, a network of numerous short cross-links on the gelatin nanostructure was constructed instead. Since glutaraldehyde readily self-polymerizes in solution, nearly monomeric glutaraldehyde (grade I) was used for consistent formation of short glutaraldehyde polymer cross-links. In addition, using grade I glutaraldehyde improved the MMP-2 cleaving rate considerably such that the release is as sensitive as previously reported MMP-2 probes. By modifying the degree of cross-linking on the gelatin nanoparticle, the QDGelNPs can be optimized to be highly responsive to MMP-2 degradation while preserving particle stability in storage, e.g., for 7 days, 14 days, 30 days, 48 days or longer.

Nanostructures

Described herein are nanostructures that are useful in the treatment and/or diagnosis of disease, e.g., cell proliferative diseases such as cancer. The term nanostructure refers to a composition that measures less than about 300-400 nm in diameter, for example about 50 nm, about 75 nm, about 100 nm, about 150 nm, about 200 nm or about 250 nm in diameter. In specific embodiments, the nanostructures of the invention are useful in the treatment and diagnosis of solid tumors. The nanostructures of the invention have the ability to change size in a tumor milieu, thereby leading to increased transport and delivery to the location of a tumor and, therefore, increased efficacy to a subject. For example, in one embodiment the nanostructures of the invention are comprised of gelatin nanostructures that contain a plurality of smaller nanoparticles that comprise a cancer therapeutic or diagnostic agent, e.g., an imaging agent. The removal/degradation of the gelatin nanostructure by proteases present in the tumor tissues allows for the release of the smaller nanoparticles.

In certain embodiments of the invention, the nanostructure comprises gelatin, as described herein.

In some embodiments, the nanostructure further comprises a charged outer surface. This charged outer surface may be comprised of peptides, carbohydrates, polymers, or small molecules that are charged, e.g., negatively or positively charged, at physiological pH. Exemplary outer surface molecules include, but are not limited to, polyethylene glycol (PEG), N-(2-hydroxypropyl)methacrylamide (HPMA), poly(vinyl-pyrrolidone) (PVP), poly(ethyleneimine)(PEI), polyamidoamines, divinyl ether and maleic anhydride (DIVEMA), dextran (alpha-1,6 polyglucose, dextrin (alpha-1,4 polyglucose), hyaluronic acid, chitosans, polyamino acids, e.g., poly(lysine) or poly(glutamic acid), poly(malic acid), poly(sapartamides), poly co-polymers, e.g., copaxone. In specific embodiments, the charged outer surface is comprised of PEG molecules. The surface groups which comprise the charged outer surface of the nanostructure can be further functionalized and bioconjugated. For example to expose a cationic surface consisting of tri-methyl ammonium end groups, an anionic surface consisting of carboxylic acid or sulfonic acid end groups, zwiterionic by exposing an amino acid, or neutral by exposing hydroxyl groups. Albumin can be conjugated to the dots as a standard platform for further conjugation and so take advantage of the extensive knowledge available regarding albumin as a conjugation scaffold for attached proteins, antibodies, or other fluorophores. For example, Tat protein (for cellular uptake) and protease cleavable peptides can be conjugated onto albumin or directly to the ends of, for example, PEG groups.

The charged outerlayer of the nanostructure is selectively removable so as to confer advantageous transport properties upon the nanostructure. For example, removal of the charged outerlayer once the nanostructure has been transported to the tumor will increase the transport of the nanostructure into the tumor. The charged outer surface can be connected to the gelatin nanostructures and/or inner nanoparticles by EDC/sulfo-NHS coupling chemistry or a linker, e.g., a cleavable peptide linker, that allows for selective removal of the charged outer surface in a desired environment. In exemplary embodiments, the inner nanoparticle core is comprised of one or more quantum dots, polymers, liposomes, silicon, silica, dendrimers, microbubbles and nanoshells. In particular embodiments the inner nanoparticle core is comprised of one or more one or more of quantum dots, polymers, liposomes, silicon, silica, dendrimers, microbubbles and nanoshells, optionally in a matrix, e.g., a PEG silicate matrix. The inner nanoparticle core may optionally comprise a ligand layer comprising one or more surface ligands (e.g., organic molecules) surrounding the core. In certain embodiments of the invention, the ligand layer may be used to couple a cleavable linker to the inner core, e.g., a peptide linker as described herein, to provide a linkage to the outer layer, and/or other inner core constituents.

Inner Nanoparticle Core Constituents

In some embodiments, the nanostructures described herein have an inner core comprising core nanoparticles, for example, one or more quantum dots, nanoshells, microbubbles, liposomes, or combinations thereof, comprising an imaging or therapeutic agent. The core nanoparticles are generally about 2-20 nm in diameter, e.g., about 5-15, about 12-14, or about 10 nm in diameter.

Quantum dots used in biological applications consist of an inorganic core, typically CdSe, that is the optically active center, an inorganic protective shell, and an organic coating designed for biological compatibility and further conjugation. In certain embodiments, the organic coating is used to conjugate a charged molecule, e.g., polyethylene glycol to the quantum dot. In a specific embodiment, the charged molecule is attached via a cleavable linker molecule. The cores are typically nearly spherical semiconductor nanostructures, ranging from about 2 to 10 nm in diameter. Core-shell quantum dots have narrow fluorescence spectra, typically about 30 nm, and quantum yields that are usually in excess of 30%. Peak positions depend both on the material and size of the quantum dot. Compared to organic dye molecules, quantum dots are particularly well suited to biological tracking, e.g., diagnostic studies, that use fluorescence as the reporter. The excitation band is very broad, requiring only that the excitation wavelength be to the blue of the emission, but the emission band is narrow and symmetric. Absorption cross sections of quantum dots can surpass those of dye molecules, especially for larger quantum dots because the distance of the extinction coefficient from the fluorescence band is proportional to the volume of the dot. For example 7.0 nm CdSe quantum dots emitting at .about.660 nm, have an extinction coefficient ranging from 1.0×10⁶ M⁻¹ cm⁻¹ at 630 nm to 6.2×10⁶ M⁻¹ cm⁻¹ at 350 nm (Leatherdale, C. A. et al. (2002) Journal of Physical Chemistry B, 106: 7619-7622). A size series of quantum dots thus represents a family of fluorophores covering a range of emission wavelengths, that are excited with the same light source, and are ideal for multiplexed detection. The accessible range of emission colors from biologically compatible quantum dots is from about 450 nm (using CdS based quantum dots) to about 800 nm (using a combination of CdSe and CdTe based quantum dots). Furthermore, because quantum dots are inorganic solids they are significantly less susceptible to photobleaching than dye molecules, making them ideal candidates for long time tracking and single molecule imaging studies.

A quantum dot will typically be in a size range between about 1 nm and about 1000 nm in diameter or any integer or fraction of an integer therebetween. Preferably, the size will be between about 1 nm and about 100 nm, more preferably between about 1 nm and about 50 nm or between about 1 nm to about 20 nm (such as about 2, 3, 4, 5, 6, 7, 8, 9, 10, 11, 12, 13, 14, 15, 16, 17, 18, 19, or 20 nm or any fraction of an integer therebetween), and more preferably between about 1 nm and 10 nm.

A core of a quantum dot may comprise inorganic crystals of Group IV semiconductor materials including but not limited to Si, Ge, and C; Group II-VI semiconductor materials including but not limited to ZnS, ZnSe, ZnTe, ZnO, CdS, CdSe, CdTe, CdO, HgS, HgSe, HgTe, HgO, MgS, MgSe, MgTe, MgO, CaS, CaSe, CaTe, CaO, SrS, SrSe, SrTe, SrO, BaS, BaSe, BaTe, and BaO; Group III-V semiconductor materials including but not limited to AlN, AlP, AlAs, AlSb, GaN, GaP, GaAs, GaSb, InN, InP, InAs, and InSb; Group IV-VI semiconductor materials including but not limited to PbS, PbSe, PbTe, and PbO; mixtures thereof; and tertiary or alloyed compounds of any combination between or within these groups. Alternatively, or in conjunction, a core can comprise a crystalline organic material (e.g., a crystalline organic semiconductor material) or an inorganic and/or organic material in either polycrystalline or amorphous form.

A nanoparticle core may optionally be surrounded by a shell of a second organic or inorganic material. A shell may comprise inorganic crystals of Group. IV semiconductor materials including but not limited to Si, Ge, and C; Group II-VI semiconductor materials including but not limited to ZnS, ZnSe, ZnTe, ZnO, CdS, CdSe, CdTe, CdO, HgS, HgSe, HgTe, HgO, MgS, MgSe, MgTe, MgO, CaS, CaSe, CaTe, CaO, SrS, SrSe, SrTe, SrO, BaS, BaSe, BaTe, and BaO; Group III-V semiconductor materials including but not limited to AlN, AlP, AlAs, AlSb, GaN, GaP, GaAs, GaSb, InN, InP, InAs, and InSb; mixtures thereof; and tertiary or alloyed compounds of any combination between or within these groups. Alternatively, or in conjunction, a shell can comprise a crystalline organic material (e.g., a crystalline organic semiconductor material) or an inorganic and/or organic material in either polycrystalline or amorphous form. A shell may be doped or undoped, and in the case of doped shells, the dopants may be either atomic or molecular. A shell may optionally comprise multiple materials, in which different materials are stacked on top of each other to form a multi-layered shell structure.

In at least one embodiment the quantum dot may optionally comprise a ligand layer comprising one or more surface ligands (e.g., organic molecules) surrounding the core. In certain embodiments of the invention, the ligand layer may be used to couple a cleavable linker to the quantum dot, e.g., a peptide linker as described herein.

Quantum dots can be chemically synthesized using wet chemical techniques that have been well described in the literature. A typical preparation consists of rapidly introducing a solution consisting of a Cd precursor, such as a cadmium carboxylate salt and a Se precursor, typically trioctylphosphine selenide (TOPSe), into a hot (>300.degree. C.) solvent mixture that contains coordinating species such as phosphonic acids, amines, and trioctylphosphine oxide (TOPO). The size of the nanocrystals obtained is precisely determined by a combination of precursor concentrations, stoichoimetric ratios, temperature, and length of reaction. Shells of ZnS or CdZnS, typically 1-2 nm thick, are grown on top of CdSe cores that have been isolated and redispersed in solutions typically consisting of mixtures of alkyl phosphines and alkyl amines. Precursors for the shell typically include diethyl zinc, dimethyl cadmium, or organic salts of Zn and Cd, and (TMS)2S. Characterization of quantum dot samples relies on Transmission Electron Microscopy (TEM) for sizing and for assessing crystal quality, UV-Vis absorption spectroscopy, and fluorescence spectroscopy for emission wavelength, linewidth, and quantum yield determination. Emission lifetimes are typically 10-25 nseconds. The spherical shape has largely been the standard for quantum dots used in biological studies, but varying this is likely a parameter that can add functionality and information. quantum dots can be grown in a variety of shapes, e.g. as nanorods with diameters <10 nm and aspect ratios as large as 10:1 or tetrapods that consist of four nanorods attached together at a central point. Varying the shape is typically achieved using combinations of alkyl phosphinic acids and by kinetically forcing the growth along the crystal axis through a large excess of precursors in solution.

For quantum dots designed for in vivo imaging or diagnostic applications, the main contributor to size is the organic coating which renders the quantum dot soluble and stable in plasma. This coating also allows peptides, e.g., peptide linker, cleavable peptides, to be covalently conjugated. The coating generally consists of an hydrophobic component that associates with the quantum dot, a hydrophilic or charged component for solubility, and a means for further conjugation (Michalet, X., et al. (2001) Single Molecules, 2: 261-276). Typically the hydrophilic component consists of PEG moieties to minimize non-specific binding and increased vascular circulation times. The important functions of the quantum dot coating are to prevent degradation of its chemical and optical properties and provide stability against agglomeration. The protective role of the organic coating is maximized by using molecules that can be cross-linked to each other, before or after their association to the quantum dot surface, to form what is effectively a poly-dentate coating unlikely to leave the quantum dot surface (through the usual dynamic binding and un-binding events typical of quantum dot-capping group associations). Further conjugation can be designed to be through the ends of the PEG chains for better accessibility, or closer to the quantum dot.

Examples of coatings for biocompatible quantum dots include organo-silica shells in which silica provides cross-linking and serves as a platform for further conjugation, ambiphilic polymers that associate hydrophobically with the native organic groups (usually TOPO) on the surface of as grown quantum dots, dendrimers, and oligomeric phosphines. Other approaches include the use of electrostatic interactions to form encapsulated particles, or the interdigitation of hydrocarbon chains, for example by using phospholipids (Dubertret, B., et al. (2002) Science, 298: 1759-1762). In all cases, conjugation to biomolecules for selective targeting is usually achieved through well-known bioconjugation techniques, such as EDC coupling with N-hydroxysuccinimides. Quantum dots can be made soluble in plasma using one of three established approaches (1) an ambiphilic polymer consisting of an acrylic acid backbone functionalized with alkyl side chains, (2) phospholipids, and (3) oligomeric phosphines that provide multiple attachment points to the quantum dot surface and expose carboxylic acid functional groups for further water compatibility and further conjugation. In all three approaches, PEGylation with varying size PEG chains allows tuning of the hydrodynamic size from 10 to 30 nm.

Clusters of quantum dots can be formed using peptide linkers. These clusters can be built so that upon cleavage by a protease, clusters break apart into individual nanostructures, so providing the possibility of decreasing the size of the probe upon exposure to the tumor environment. Clusters of quantum dots can be synthesized with two colors so as to be able to observe the break-up in vivo through a sudden change in the spectrum of the probe, or at the single dot level the disappearance of one of the two colors. If the quantum dots are close enough for efficient energy transfer within the clusters, it is likely that the redder of the two colors will dominate while the cluster is intact, with both colors observed upon break-up. This can be monitored with in vitro FRET characterization experiments.

The inner core may also contain one or more nanoshells, e.g., metal nanoshells. Nanoshells are nanoparticles comprised of a dielectric core surrounded by an ultra-thin metal and characterized by highly tunable optical resonances (Hirsch et al. (2006) Annals of Biomedical Engineering 34:15-22). Nanoshells have been shown to effectively kill tumor cells when used in conjunction with near-infrared light (Hirsch et al. (2003) PNAS 100:13549-13554).

The inner core can also contain one or more microbubbles. Microbubbles comprise a water insoluble gas surrounded by a biological layer, e.g., a lipid layer. Synthetic microbubbles have been developed and are useful imaging agents due to their altered reflectivity of ultrasound energy (Feinstein et al. (2004) Am J Phsyiol Heart Circ Physiol. H450-7). In addition to the ability of microbubbles to be used as imaging agents, they can also be used to deliver therapeutics by, for example, conjugating biological or chemical moieties to the biological layer (Klibanov et al. (2006) Investigative Radiology 41:354-62).

The inner core can contain one or more liposomes. Liposomes are microscopic phospholipid bubbles with a bilayerd membrane that have been shown to be effective for delivering therapeutic and imaging agents (for a review see, Torchilin, V. (2005) Drug Discovery 4:145-160). The half-life of liposomes can be extended by protecting them with, for example, polyethylene glycol, poly[N-(2-hydroxylpropyl)methacrylamide], poly-N-vinylpyrrolidones, L-amino-acid-based biodegradable polymer-lipid conjugates or polyvinyl alcohol, thus increasing their utility for the delivery of therapeutic or imaging agents. A number of liposomes are currently marketed. For example, Doxil® is a pegylated Liposomal composition containing doxorubicin used for the treatment of cancer (Gabizon et al. (2003) Clin. Pharmacokinet 42:419-36).

The inner core materials of the invention disclosed herein, e.g., therapeutic and imaging agents, can be combined within a single nanostructure to provide beneficial treatment or diagnostic effects. For example, the nanostructures of the invention may contain one or more anticancer agents, i.e., cancer therapeutic agents. The anticancer agents may be formulated into the inner core of the nanostructure, e.g., into a polymer matrix. Alternatively, the therapeutic agent may be formulated into material that is used to formulate the inner core of a nanostructure comprising multiple inner core constituents (e.g., quantum dots). In another embodiment, the anticancer agent may be conjugated to the coating of the inner core, e.g., of a quantum dot, so as to be covalently, or non-covalently attached to the inner core after cleavage of the charged layer.

Exemplary cancer therapeutic agents include chemical or biological reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990) and Teicher, B. A. Cancer Therapeutics: Experimental and Clinical Agents (1996) Humana Press, Totowa, N.J.), and are typically used to treat neoplastic diseases. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

In alternative embodiment, the nanostructure may be used to deliver an imaging agent, i.e., a detectable label, to a tumor. The detectable label can be directly detectable (i.e., one that emits a signal itself). Alternatively, the detectable label can be indirectly detectable (i.e., one that binds to or recruits another molecule that is itself directly detectable, or one that cleaves a product to generate directly detectable substrates). Generally, the detectable label can be selected from the group consisting of an electron spin resonance molecule (such as for example nitroxyl radicals), a fluorescent molecule, a chemiluminescent molecule, a radioisotope, an enzyme, an enzyme substrate, a biotin molecule, an avidin molecule, a streptavidin molecule, a peptide, an electrical charge transferring molecule, a colloid gold nanocrystal, a ligand, a microbead, a magnetic bead, a paramagnetic particle, a chromogenic substrate, an affinity molecule, a protein, a peptide, a nucleic acid, a carbohydrate, an antigen, a hapten, an antibody, an antibody fragment, and a lipid. In specific embodiments, gadolinium, manganese and iron may be used as detectable labels for MRI; iodine may be used for X-ray/CT; and low density lipids and gas microbubbles in a stabilizing shell for ultrasound.

The surface of the inner core can be conjugated with a cleavable peptide linker, e.g., a protease cleavable linker, that provides a linkage to the outer layer or to other inner core constituents. Exemplary proteases useful in the methods of the invention include, but are not limited to: Cathepsin B, Cathepsin D, MMP-2, Cathepsin K, Prostate-specific antigen, Herpes simplex virus protease, HIV protease, cytomegalovirus protease, thrombin, and interleukin 1.beta. converting enzyme. Moreover, peptides that comprise the protease recognition sequence of theses proteases are useful in the methods and compositions of the invention (for details regarding the sequence of protease cleavable linkers see, for example, Mahmood et al. (2003) Molecular Cancer Therapeutics 2:489-96). In specific embodiments, linkers useful in the methods of the invention include thrombin cleavable linkers (see, ChemBioChem 3:207-211, 2002), Cathepsin cleavable linkers (see, Bioconjugate Chem 9: 618-626), MMP-2 cleavable linker (see JBC 265: 20409-20413, 1990), HIV protease cleavable linker (see, Bioorganicheskaia Khimiia 25:911-922, 1999), acid cleavable linkers (see, Crit. Rev Drug Carrier Syst 16:245-288, 1999), and photo cleavable linkers, e.g., those available from Novabiochem).

The peptide linker can itself be conjugated to render it, for example, cationic (with trimethyl ammonium or anionic with carboxylic acid or sulfonic acid). Upon cleavage, the particles can then switch potential from cationic to anionic or vice versa as follows. The cleavable peptide sequence can be coupled to terminal amine or to carboxylic acid groups using established conjugation chemistries. Unconjugated carboxylic acid (amine) groups provide negative (positive) charge which is balanced by the cationic (ionic) charge conjugated to the peptide. Upon cleavage, the net charge of the nanostructures switches from cationic to anionic or vice versa. The peptide linker can also be functionalized with a fluorescent dye, such as a rhodamine based conjugate. In specific embodiments, the quantum dot can serve as an efficient FRET acceptor if the dye emission overlaps with the absorption of the quantum dot, and, if the two are close enough. Upon cleavage, fluorescence from the dye will be observed if FRET is efficient. If FRET is not efficient, the quantum dot-linker-dye complex will co-localize emission from the two colors, while upon cleavage the position of the two colors will be distinct. In vitro FRET control experiments will be used to characterize these complexes.

Methods of Treatment

The term “subject” is intended to include organisms, e.g., prokaryotes and eukaryotes, which are capable of suffering from or afflicted with a cell proliferative disorder, e.g., cancer. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer.

The term “neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli that evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histology and other features of a neoplasm, it can be determined whether the neoplasm is benign or malignant. Invasion and metastasis (the spread of the neoplasm to distant sites) are definitive attributes of malignancy.

Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. In contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue, may be converted to a fibrous connective tissue capsule allowing for easy surgical removal of the benign tumor.

Conversely, malignant tumors are locally invasive and grow into the adjacent tissues usually giving rise to irregular margins that are not encapsulated making it necessary to remove a wide margin of normal tissue for the surgical removal of malignant tumors. Benign neoplasms tend to grow more slowly and tend to be less autonomous than malignant tumors. Benign neoplasms tend to closely histologically resemble the tissue from which they originated. More highly differentiated cancers, i.e., cancers that resemble the tissue from which they originated, tend to have a better prognosis than poorly differentiated cancers, while malignant tumors are more likely than benign tumors to have an aberrant function, e.g., the secretion of abnormal or excessive quantities of hormones.

The term “cancer” includes malignancies characterized by deregulated or uncontrolled cell growth, for instance carcinomas, sarcomas, leukemias, and lymphomas. The term “cancer” includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor.

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. The term “adenocarcinoma” includes carcinomas derived from glandular tissue or a tumor in which the tumor cells form recognizable glandular structures.

The term “sarcoma” includes malignant tumors of mesodermal connective tissue, e.g., tumors of bone, fat, and cartilage.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary-carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include vaginal, cervical, and breast cancers.

The language “inhibiting growth,” as used herein, is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by dysregulated endothelial cell division.

The language “inhibiting tumor growth” or “inhibiting neoplasia” includes the prevention of the growth of a tumor in a subject or a reduction in the growth of a pre-existing tumor in a subject. The inhibition also can be the inhibition of the metastasis of a tumor from one site to another. In particular, the language “tumor” is intended to encompass both in vitro and in vivo tumors that form in any organ or body part of the subject. Examples of the types of tumors intended to be encompassed by the present invention include those tumors associated with breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidneys. Specifically, the tumors whose growth rate is inhibited by the present invention include basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas (i.e. maglinant lymphomas, mantle cell lymphoma), malignant melanomas, multiple myeloma, epidermoid carcinomas, and other carcinomas and sarcomas.

The language “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and Teicher, B. A. Cancer Therapeutics: Experimental and Clinical Agents (1996) Humana Press, Totowa, N.J. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

The instant invention provides therapeutic methods for the treatment of a subject having cancer. In certain embodiments, a subject is administered a nanostructure of the invention to alleviate one or more symptoms of cancer. For example, a subject can be administered a nanostructure of the invention to reduce the size or eliminate a solid tumor.

The dosages administered will vary from patient to patient; a “therapeutically effective dose” can be determined, for example, by monitoring the size or growth rate, or the duration of the growth period of a tumor, tumor number, cancer cell number, viability, growth rate and the duration of the growth period of a cancer cell. A therapeutically effective dose refers to a dose wherein the combination of compounds has a synergistic effect on the treatment of cancer.

In the treatment of cancer, a therapeutically effective dosage regimen should be used. By “therapeutically effective”, one refers to a treatment regimen sufficient to decrease tumor size or tumor number, decrease the rate of tumor growth or kill the tumor. Alternatively, a “therapeutically effective regimen” may be sufficient to arrest or otherwise ameliorate symptoms of the cancer. Generally, in the treatment of cancer, an effective dosage regimen requires providing the medication over a period of time to achieve noticeable therapeutic effects. The pharmaceutical composition may be formulated from a range of preferred doses, as necessitated by the condition of the patient being treated.

The loading capacity per particle for a 10-nm nanocarrier may be limited. Therefore, not only the penetration depth but also sufficient quantity of nanostructures should be delivered.

### EXAMPLES

The invention is further described in the following examples, which do not limit the scope of the invention described in the claims.

The following reagents were used in these examples. Gelatin type A ˜175 bloom from porcine skin, glutaraldehyde solution (Grade I, 50%), N-Hydroxysulfosuccinimide Sodium salt (sulfo-NHS), N-(3-Dimethylaminopropyl)-N′-ethylcarbodiimide hydrochloride (EDC), methoxypolyethylene glycol amine 5,000 (mPEG-amine 5 kDa), agarose, hydrochloric acid fuming 37% TraceSelect, Nitric Acid >69.0% TraceSelect, and Ethylenediaminetetraacetic Acid, Disodium Salt Dihydrate (EDTA) were purchased from Sigma-Aldrich. Acetone, HEPES, and 10×PBS Liquid Concentrate were purchased from EMD Chemicals Inc. (Gibbstown, N.J.). Calcium chloride, isopropyl alcohol, and glycine were obtained from Mallinckrodt Baker Inc. (Phillipsburg, N.J.). ICP-OES cadmium standard 2% HNO3 1,000 mg/L was purchased from Perkin Elmer (Shelton, Conn.). Qdot® 565 ITK™ amino (PEG) quantum dot and GIBCO® Certified Heat-Inactivated Fetal Bovine Serum was obtained from Invitrogen (Eugene, Oreg.). Block™ Casein in PBS was purchased from Thermo Scientific (Rockford, Ill.). Reagent grade deionized water used for ICP-OES experiments was purchased from Ricca Chemical Company (Arlington, Tex.). Water for all other experiments was obtained using a Barnstead NANOpure® DIamond Life Science UV/UF TOC water system (Thermo Fisher Scientific, Suwanee, Ga.). Active Human Recombinant MMP-2 was purchased from EMD Chemicals Inc. (Gibbstown, N.J.).

### Example 1

### Multistage Particle Synthesis

The multistage quantum dot gelatin nanostructures (QDGelNPs) are composed of a gelatin core with amino-PEG QDs conjugated to the surface using EDC/sulfo-NHS coupling chemistry. Another layer of 5 kDa PEG is conjugated to the surface of the gelatin nanoparticles to confer long blood circulation time.

Gelatin nanostructures were prepared from a modification of the two-step desolvation method developed in (Netti P A, Berk D A, Swartz M A, Grodzinsky A J, & Jain R K (2000), Cancer Research 60(9):2497-2503). Gelatin type A (0.625 g) was added to 12.5 mL of DI water and heated at 40° C. until dissolution. The solution was then quickly removed from heat and 12.5 mL of acetone was added to the solution at 6.0 mL/min while stirring at 300 rpm. After the acetone addition was complete, the stirring was turned off. After exactly 1 min, the supernatant containing the low molecular weight gelatin fraction was removed. DI water (12.5 mL) was added to the remaining precipitate and heated again to 40° C. until dissolution. Half the solution was removed and the pH of the remaining half was adjusted to 2.7 with a 1 M HCl solution. Under constant stirring at 600 rpm and 40° C., 20.75 mL of acetone was added at 1 mL/min. Following addition of ˜17 mL of acetone, the solution appeared cloudy white from the scattering by gelatin particles being formed. After the acetone addition is complete, 30 uL of 50% glutaraldehyde solution (grade I) diluted in 1 mL acetone was added to the gelatin solution at 0.05 mL/min to cross-link the particles. Subsequently, the solution was kept at 40° C. and 600 rpm stir rate for 7.5 hours. The acetone was then rotavapped off slowly until a final volume of 5-6 mL. The remaining solution was filtered through a 0.2 μm syringe filter. A 1 M glycine solution (0.2 mL) was added and the solution was stored overnight at 4° C. Instead of centrifugation to purify the nanostructures, GFC were used to prevent aggregation and an increase in size. A 1 mL solution of the gelatin nanostructures was injected into a Superose™ 6 GL 10/300 column (GE Healthcare, Piscataway, N.J.) with 1×PBS as the mobile phase. The peak eluting at the void volume were collected with 0.5 mL fractions. This was repeated once more and the first concentrated fractions from both GFC runs were combined. This 1 mL solution was then taken to the next step for QD conjugation and PEGylation.

Synthesis of QDGelNP was performed as follows. The 1 mL of gelatin nanostructures was combined with 20 uL of 8 uM Qdot® 565 ITK™ amino (PEG) QDs, and the solution was stirred at 130 rpm for 30 minutes. Afterwards, the pH was changed to 5 and then immediately adjusted to pH 6. Stirring continued for 30 minutes. EDC (0.4 mg, 2.1 μmol) and sulfo-NHS (0.4 mg, 1.9 μmol) was dissolved in 50 μL of DI water and then added to the gelatin nanostructure/QD mixture. The reaction proceeded for 3 hours. Afterwards, a solution of mPEG amine 5 kDa (20 mg, ˜4 μmol) dissolved in 50 μL of DI water was added to the gelatin/QD solution. Then an additional solution of EDC (0.4 mg) and sulfo-NHS (0.4 mg) dissolved in 50 μL of DI water was added. After two hours, the pH was adjusted to 8 and stirring continued for 1 hour. A 1 M glycine solution (50 μL) was added to quench the reaction. After 30 minutes, the resulting mixture was filtered through a 0.2 μm syringe filter and then purified using GFC with the Superose™ 6 column. The mobile phase used was 50 mM HEPES buffer at pH 7.5 for subsequent in vitro characterization of the QDGelNPs or 1×PBS at pH 7.4 for subsequent in vivo experiments. The peak eluting at the void volume was collected with 0.5 mL fractions and the first concentrated fraction was used for further experiments.

X-ray photoelectron spectroscopy (XPS) performed as follows was used to determine the presence of PEG chains on the QDGelNPs surface. QDGelNPs with and without PEGylation were transferred to DI water by centrifugation at 10,000 rpm for 10 min and washed with DI water three times. The particles were then drop-casted on a p-type silicon substrate (University Wafer, South Boston, Mass.) and dried overnight in vacuum. The samples were characterized using a Kratos AXIS Ultra Imaging X-ray Photoelectron Spectrometer (Kratos Analytical, Chestnut Ridge, N.Y.) with a monochromatized Al Kα X-ray source. High resolution analysis of the C1s spectra was performed to determine the chemical composition from the hydrocarbon (C—C or C—H at 285.0 mV), ether (C═O at 286.4 mV), carbonyl (C═O at 288.2 mV), and carboxyl (C═O at 289.1 mV) envelops. Deconvolution of the spectra determined the area under the peak for each chemical species and thus their relative composition.

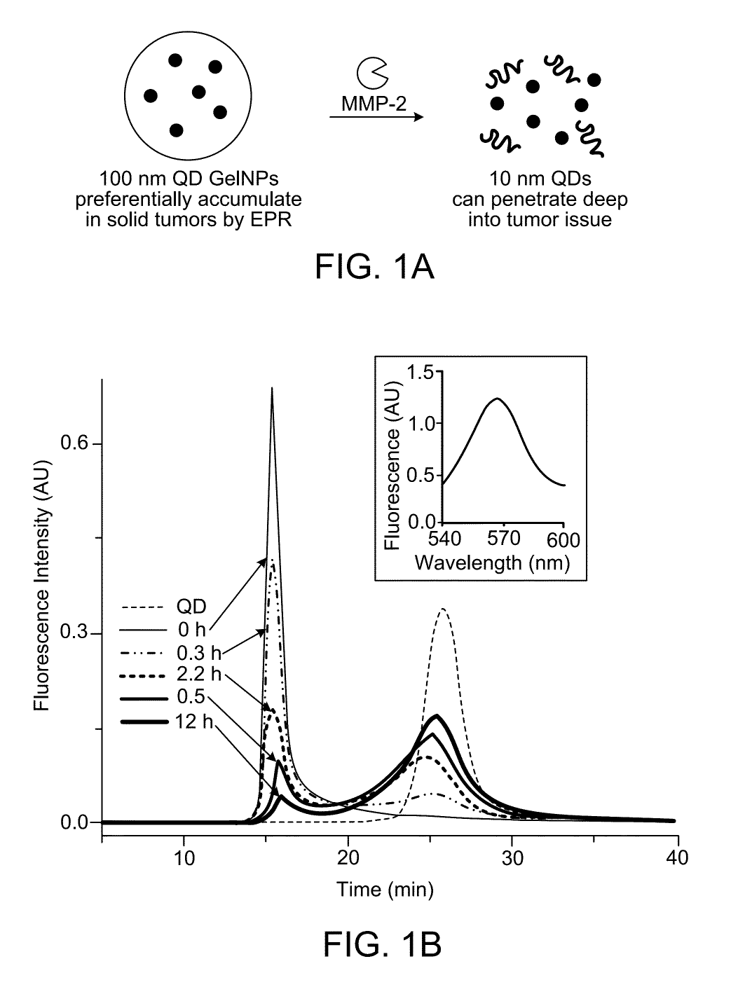

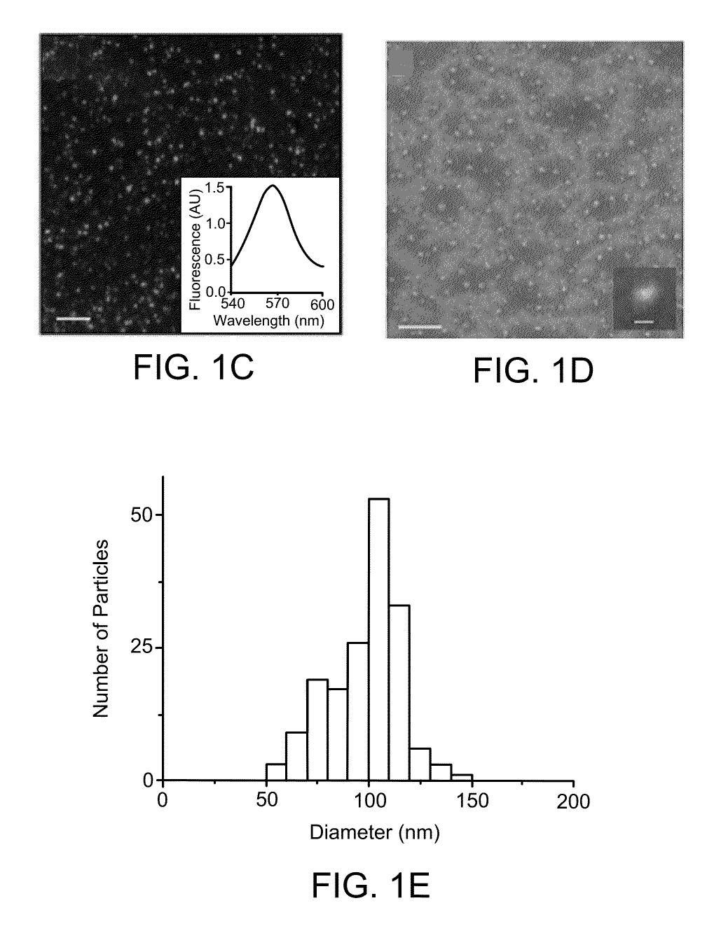

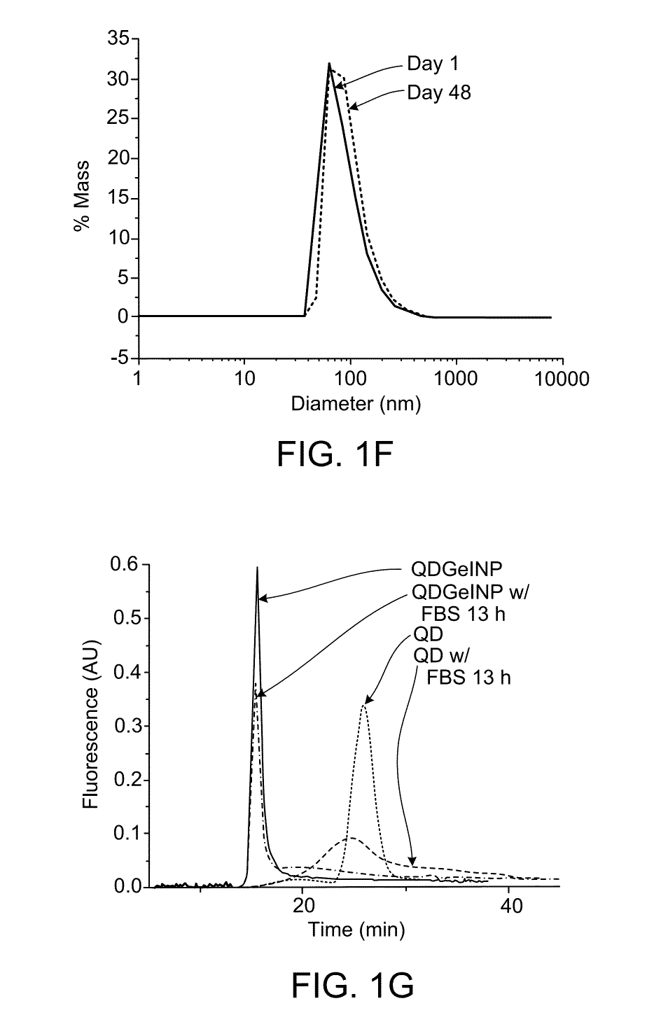

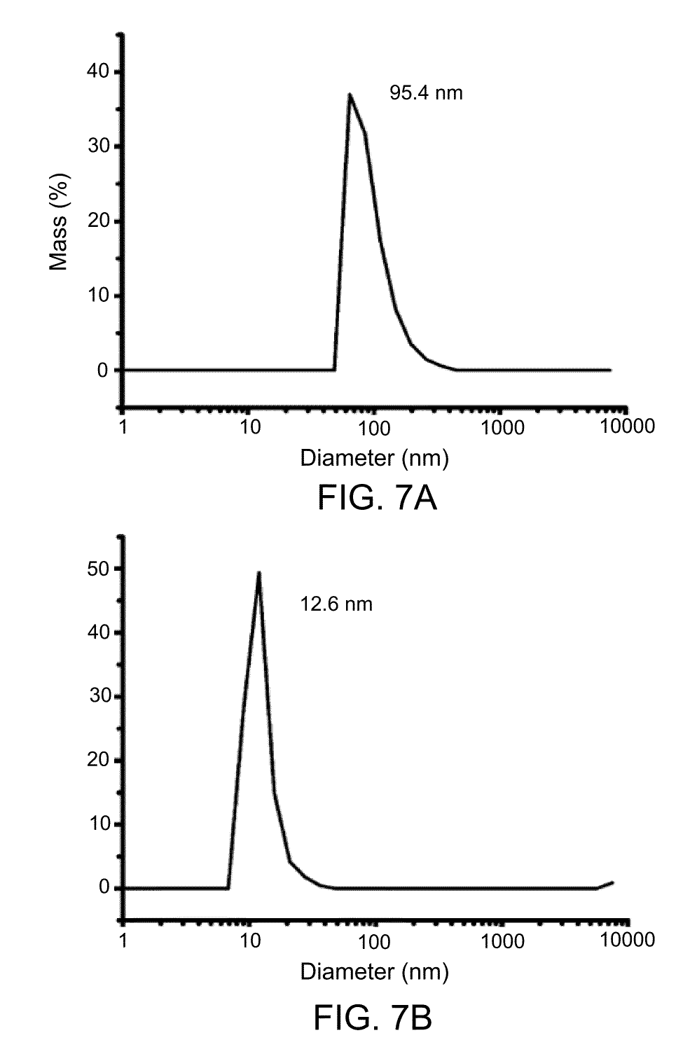

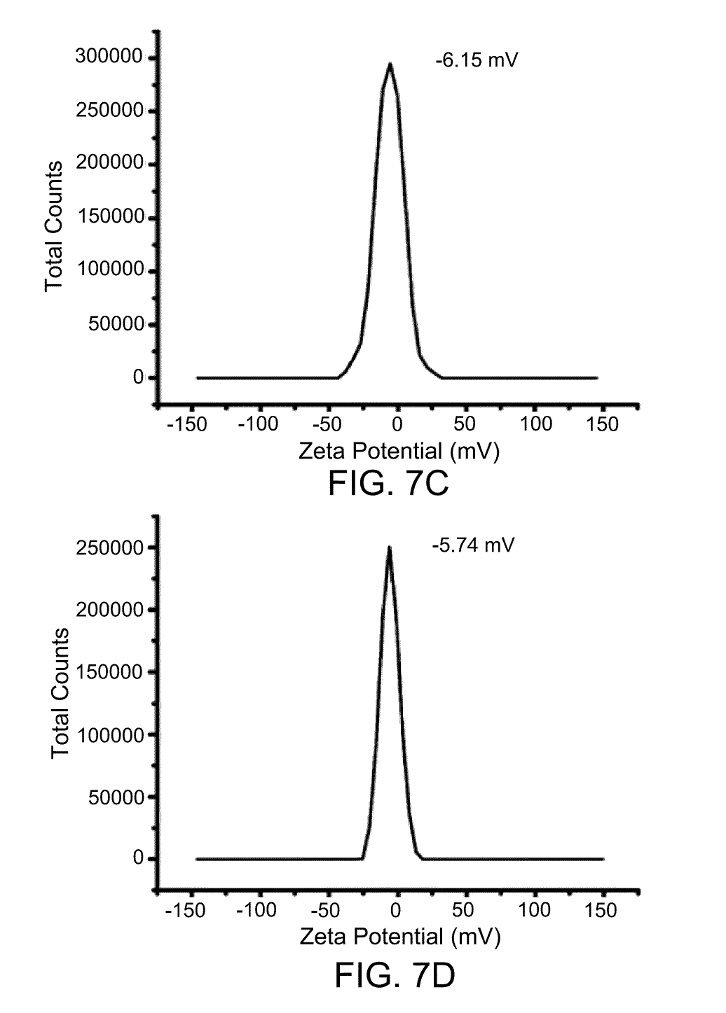

Dynamic light scattering (DLS) of the QDGelNPs before and after PEGylation (but before purification) was performed as follows. The measurements of hydrodynamic diameter were carried out on a DynaPro Titan Dynamic Light Scatterer in pH ˜7.5 50 mM HEPES buffer at 25° C. The result is the average of five measurements of 10 acquisitions each. The results indicate an increase in diameter from 78.3±0.2 nm to 93.7±0.5 nm. DLS of the final structure after purification and size selection using gel filtration chromatography (GFC) revealed a single particle distribution with a hydrodynamic diameter of 97.9±2.1 nm and a polydispersity of 41.2% (FIGS. 7A-D). This value agreed well with the average nanostructure diameter of 99±1 nm estimated from scanning electron microscopy (SEM) (FIGS. 1D-E). SEM samples were prepared by desalting the nanostructure suspension two times using GFC with DI water as the mobile phase. Then a 1:1 mixture of the resulting nanoparticle suspension in DI water and acetone was drop-casted on a cleaned p-doped silicon substrate (University Wafer, South Boston, Mass.). The sample was dried in air overnight, and then the surface was sputter coated with ˜3 nm of gold-palladium. Images were taken on a FEI/Philips XL30 field emission gun environmental scanning electron microscope. Size analysis was performed by manually measuring 170 particles using ImageJ.

Inductively coupled plasma optical emission spectroscopy (ICP-OES) determined a concentration of 15 pmol of QDs per mg of QDGelNPs. ICP-OES analyses were performed as follows. The concentration of QD in a QDGelNP sample was determined by measuring its Cd content and then comparing it to the Cd content in a QD sample of known concentration. The QDGelNP and QD standard solutions were first completely dissolved in aqua regia (HCl:HNO3=3:1) solution, and then its Cd content was determined using a Horiba Jobin Yvon Activa ICP-OES instrument. QDGelNP in solution (300 uL) was heated to 130° C. in a glass vial until the water has completely evaporated. Fresh aqua regia solution (250 μL) was added to the dried QDGelNP sample. The solution was covered with parafilm and sonicated for 5 minutes. Afterwards the solution was heated overnight at 65° C. DI water (1.75 mL) was added the next day using VWR® Labmax™ Bottle-Top Dispenser. Samples made from cadmium ion calibration standards and known amount of QDs were prepared by the same method. These solutions were prepared so that their Cd concentration would fall above and below the QDGelNP sample's Cd concentration. The calibration was linear with an error of less than 2%. The ICP-OES intensity was the average of five (30 sec) exposures. A calibration curve for the amount of QD was created using the QD standard solutions to give a value for the mole of Cd per mole of QD. This value was used to determine the amount of QD in the QDGelNP solution. The QDGelNPs showed excellent colloidal stability; their diameter by DLS remained nearly unchanged while in storage at ˜4° C. over 48 days—from 95.7±4.1 nm on day 1 to 101.1±2.5 nm on day 48 (FIG. 1F and Table 1).

TABLE 1
Mean value and S.E.M. of five DLS size measurements for QDGelNP on
day 1 and day 48 after synthesis and storage at 4° C.

| | Day 1 | Day 48 |
|---|---|---|
| Diameter (M ± SEM, nm) | 95.7 ± 4.1 | 101.1 ± 2.5 |
| Percent Polydispersity | 53.4 ± 3.8 | 50.9 ± 2.4 |
| (M ± SEM, %) | | |

Both the initial 100-nm nanostructures and the released 10-nm NPs were designed to have a neutral surface charge, ensuring the difference in transport before and after cleaving is only a result of size change. In addition, previous report suggested neutral particles are optimal for diffusion in the interstitial matrix (Stylianopoulos T, et al. (2010), Biophysical Journal 99:4). Measurements of nanoparticle ζ-potential were carried out on a Malvern Instruments ZetaSizer ZS90 with the Universal Dip Cell at 25° C. For the measurements, 20 μL of nanostructure suspension was dispersed in 800 μL of 50 mM HEPES buffer at either pH ˜7.5 and ˜6.0. The result is the average of five measurements of 20 acquisitions each. The ζ potential of the ˜100 nm QDGelNPs at pH 7.5 was −6.29±0.22 mV and at pH 6 was −5.00±0.12 mV. The ζ potential of the ˜10 nm QDs used for the second stage NP was −5.13±0.16 mV at pH 7.5 and −4.36±0.17 mV at pH 6 s (FIG. 7A-D). These results confirm the charge neutrality of both particles in the pH range found in normal tissues and solid tumors.

### Example 2

### MMP-2 Triggers a Size Change

Next the ability of MMP-2 to change the size of QDGelNPs was investigated in vitro using GFC. GFC chromatograms using fluorescence detection (ex: 250 nm, em: 565 nm) were obtained from incubation of 0.1 mg of QDGelNPs with 235 ng (0.16 μM) of MMP-2 at 37° C. (FIG. 1B). Previous report have estimated the extracellular concentration of MMP-2 in HT-1080 (human fibrosarcoma) xenograft tumor tissue in vivo at approximately 1 mM (Hatakeyama H, et al. (2007), Gene Ther 14(1):68-77), significantly higher than the concentration used in the in vitro experiment.

GFC was performed on the QDGelNP before and after incubation with MMP-2 for various lengths of time. Four samples of 0.1 mg QDGelNP in 50 mM HEPES, 2 mM CaCl2 were incubated with 235 ng of MMP-2 for 0.25, 2.25, 5, or 12 hours at 37° C. At the end of the incubation time, EDTA solution was added to give a final concentration of 20 mM EDTA to inhibit further MMP-2 degradation. The samples before and after cleaving with MMP-2 were directly analyzed by GFC with a Superose™ 6 GL 10/300 column (GE Healthcare, Piscataway, N.J.) on an Agilent 1100 series HPLC with an in-line degasser, autosampler, diode array detector, and fluorescence detector (Roseville, Calif.). The injection volume was 45 μL and the mobile phase was 1×PBS (pH 7.4) at a flow rate of 0.5 mL/min. The GFC chromatograms were detected by fluorescence detection at 250 nm excitation wavelength and 565 nm emission wavelength, allowing us to measure only the elution profile of the QDs. Due to changes in the QD fluorescence intensity over time and scattering effects, the chromatograms integrated intensities from 13 to 38 min were normalized to unity. The percent of QDs that were released over time was then analyzed. The peak corresponding to the free QDs was integrated (from 18-38 min) and then corrected for peak tailing from the peak at void volume. A simple peak tailing correction was done by assuming that for a certain integrated area of the peak at void volume, it will add a fixed percentage of that area to the peak for free QDs. This fixed percentage (26.5%) was obtained using QDGelNP before cleaving, which should elute completely at the void volume if not for peak tailing.

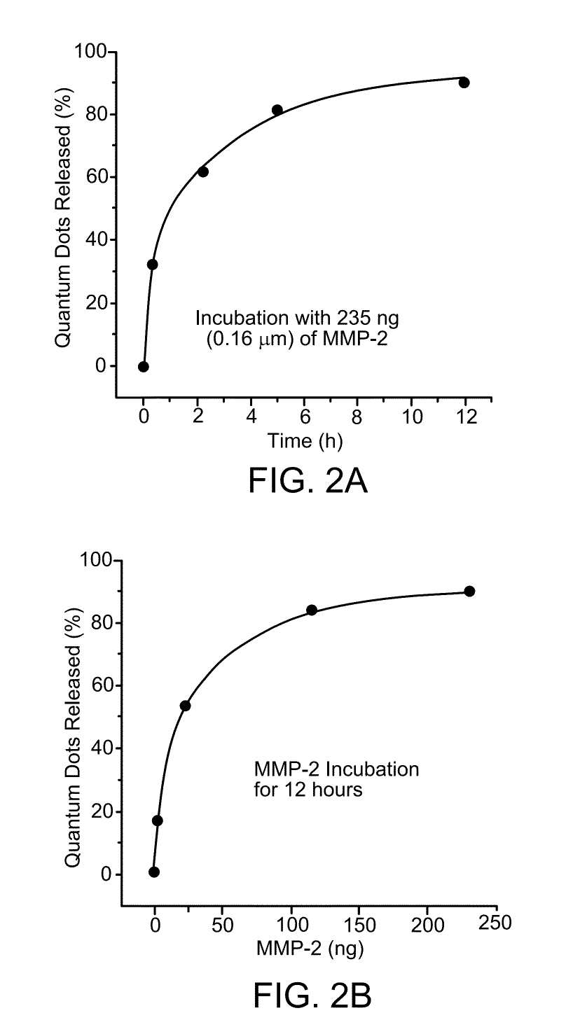

The QDGelNPs initially eluted at the GFC column's void volume but after incubation with MMP-2 for various times up to 12 hours, the peak shifted to a longer elution time corresponding to the smaller size of individual QDs, whereas incubation with 50% fetal bovine serum (FBS) showed no such shift (FIG. 1G). 50% of the QDs were released in ˜1.5 hours and the percent of freed QDs saturated at ˜90% (FIG. 2A), regardless of longer incubation times or addition of more MMP-2. This experiment was repeated with the incubation time kept constant at 12 hours but the amount of MMP-2 was varied (FIG. 2B). Under this condition, only ˜25 ng of MMP-2 was necessary to release 50% of the QDs. These results demonstrated the MMP-2 triggered size change occurred in an efficient and complete manner.

### Example 3

### Diffusion of Payload

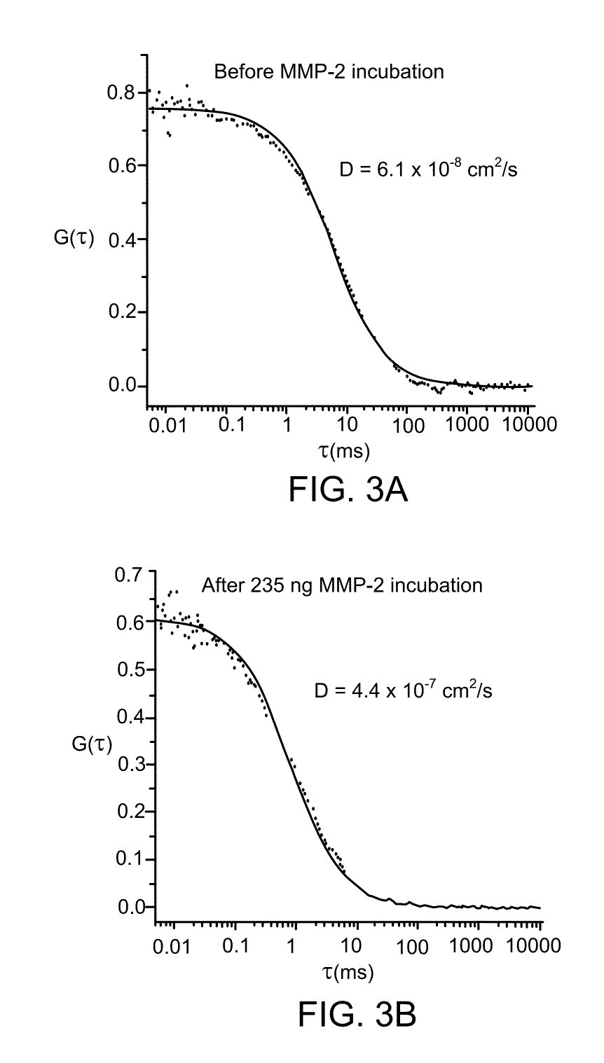

The experiments described in this example were performed to verify that the released QDs diffuse optimally in the interstitial matrix, that residual gelatin/glutaraldehyde on their surface, imparted by cleavage of the gelatin nanostructures, would not lead to extraneous binding interactions or significant size increase. To investigate this possibility, fluorescence correlation spectroscopy (FCS) was used to directly measure the hydrodynamic diameter/diffusion coefficient of the QDs before and after cleaving the gelatin core (FIGS. 3A-B).

FCS was performed as follows. Fluorescence correlation spectroscopy measurements were carried out on a custom-built inverted confocal microscopy setup equipped with a Nikon Plan Apo VC (60×, 1.2 N.A.) water immersion objective. The 514 nm laser line from an argon-krypton-ion laser (Innova 70C, Coherent Inc., Santa Clara, Calif.) at 6.2 μW (measured at the back aperture of the objective) was used for excitation. The emission was collected through the same objective and spatially filtered by focusing through a 25 μm pinhole. Following a 550 nm long-pass filter, the fluorescence was split using a 50-50 beamsplitter and recorded using avalanche photodiodes (SPCM-AQR-13, Perkin Elmer, Shelton, Conn.). Cross-correlation was performed using a digital correlator (ALV7004-FAST, ALV, Langen, Germany) and correlogram fitting was performed using the ALV software. Sample carriers were formed by sealing a silicone perfusion chamber (PC8R-2.5, Grace Bio-Labs Inc., Bend, Oreg.) over a 22×50 mm glass coverslip (VWR, Pittsburgh, Pa.). The cover slip surface was pretreated with casein/PBS solution to prevent nonspecific binding. HEPES (50 mM, pH 7.5) was used as the solvent. The QDGelNP suspension was diluted to ˜0.2 mg/mLs. The temperature during the measurement was ˜296° K. Five to ten measurements with acquisition times of 60 seconds each were performed for each sample.

FCS Analysis was performed as follows. In the confocal setup, the axial dimension of the focal volume is significantly larger than the lateral dimensions. Hence, it is sufficient to fit the cross-correlation function using the isotropic 2D translational diffusion model:

$$g ( τ ) = \frac{1}{{N}_{p}} \frac{1}{( 1 + \frac{τ}{{τ}_{D}} )} .$$

This allows us to obtain the correlation time for each measurement. Then the diffusion coefficient was calculated using the equation:

$$D = \frac{{ω}^{2}}{4 {τ}_{D}} .$$

The radius of the confocal detection volume (w) was obtained using 0.10 urn calibration standards (R100, Thermo Scientific) and the QDs. Diffusion coefficients from repeated measurements were averaged. Hydrodynamic radius was obtained from the mean diffusion coefficient using the Einstein-Stokes equation:

$$D = \frac{{k}_{B} T}{6 π η r} .$$

The value used for viscosity (η) of water at 295.6° K was 9.4×10-4 Pa·s.

The hydrodynamic diameter by FCS before cleaving was 81.1±2.3 nm (D=5.6±0.2×10⁻⁸ cm² s⁻¹), which is consistent with the DLS measurement of 90.9±1.3 nm for this batch. After MMP-2 digestion, the hydrodynamic diameter decreased to 9.7±0.3 nm (D=4.7±0.2×10⁻⁷ cm² s⁻¹), which is the size of individual QDs, indicating the size increase of the released QDs from gelatin/glutaraldehyde fragments was negligible.

Next, whether the size change observed in GFC and FCS enhances diffusive transport was evaluated in dense collagen environments resembling those in solid tumors. To simulate the interstitial matrix of a solid tumor, a collagen gel was prepared in a capillary tube at 0.74% (7.4 mg/mL) concentration, similar to the reported estimate of 9.0±2.5 mg/(mL interstitial matrix) for interstitial collagen in both human colon adenocarcinoma (LS174T) and murine mammary carcinoma (MCalV) implanted in mouse dorsal chambers (Ramanujan S, et al. (2002), Biophysical Journal 83(3):1650-1660; Netti P A, Berk D A, Swartz M A, Grodzinsky A J, & Jain R K (2000), Cancer Research 60(9):2497-2503), as follows.

Collagen hydrogels were prepared by mixing the following components in order on ice: 141.75 μL of 8.6 mg/ml rat tail collagen I (354249, BD Biosciences), 3.8 μL, of 1 N sodium hydroxide, and 19.5 μL of 0.17 M EDTA. The final concentration of collagen was 7.38 mg/ml and EDTA was 20 mM. After vortexing, the gel was added to partially fill a microslide capillary tube (Vitrocom Inc. No. 2540, Mt. Lakes, N.J.), then incubated overnight at 37° C. QDGelNPs (0.1 mg) was incubated with 235 ng of activated MMP-2 for 12 hours in 50 mM HEPES 2 mM CaCl₂. At the end of 12 hours, EDTA was added to give a final concentration of 20 mM. A 20 μL mixture of the QDGelNPs either before or after incubation with MMP-2 and SilicaQDs was added into the capillary tube and in contact with the surface of the collagen gel. The concentration of the two particles and sensitivity of the avalanche photodiodes (APD) were adjusted so that both particles gave similar signal intensities. The sample was left in ˜295° K for 12 hours and then imaged using a multiphoton laser scanning microscope. Image analysis was performed using ImageJ. The concentration profile for the QDGelNPs after cleaving was fitted to the following one-dimensional model to obtain the diffusion coefficient in the collagen gel (Clauss M A & Jain R K (1990), Cancer Research 50(12):3487-3492):

$$C ( x , t ) α erfc ( \frac{x}{2 \sqrt{{D}_{eff} t}} ) ,$$

where erfc is the complementary error function. The nonlinear curve fitting was performed using fminsearch in Matlab®. The diffusion coefficient ratio (D/Dₒ) was compared to reported values in Ramanujan S, et al. (2002), Biophysical Journal 83(3):1650-1660 and Stylianopoulos T, et al. (2010), Biophysical Journal 99:4. In Stylianopoulos T, et al. (2010), Biophysical Journal 99:4, D/Dₒ was found to be ˜0.35 for an 11.2 nm QD in 9.37 mg/mL collagen gel. In Ramanujan S, et al. (2002), Biophysical Journal 83(3):1650-1660, a value of ˜0.95 for D/Dₒ was obtained for a ˜10 nm particle in 2.4 mg/mL collagen gel. Values for D/Dₒ obtained for higher concentrations of collagen gel from Ramanujan S, et al. (2002), Biophysical Journal 83(3):1650-1660 were not used because their concentrations were prepared by centrifuging low-concentration gels and they did not match gels prepared directly from high-concentration solutions. By simple linear interpolation of these two values, a D/Dₒ for ˜10 nm NP in 7.38 mg/mL collagen of 0.52 was obtained.

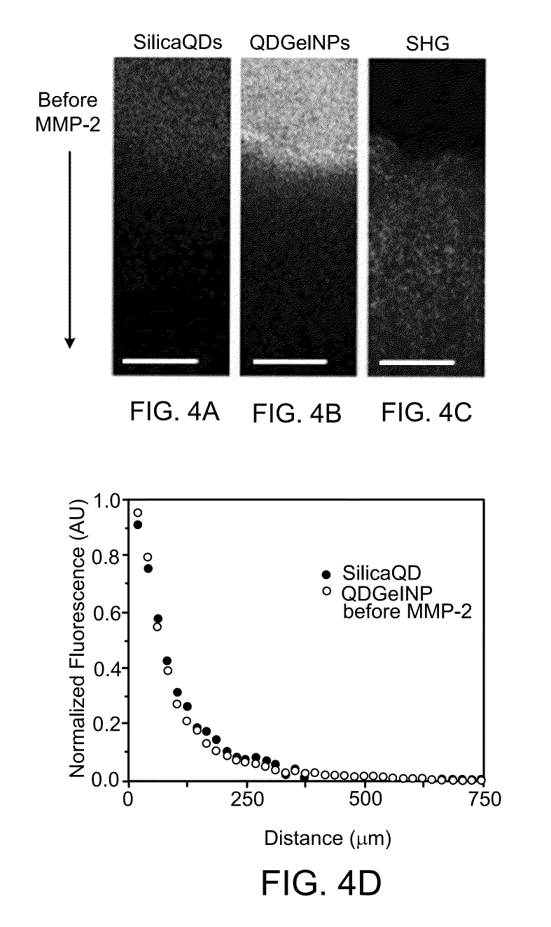

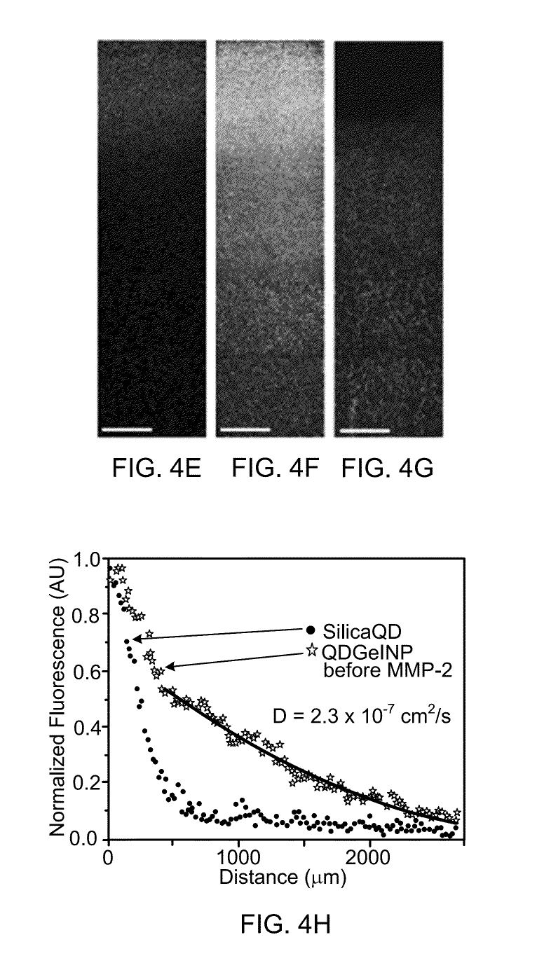

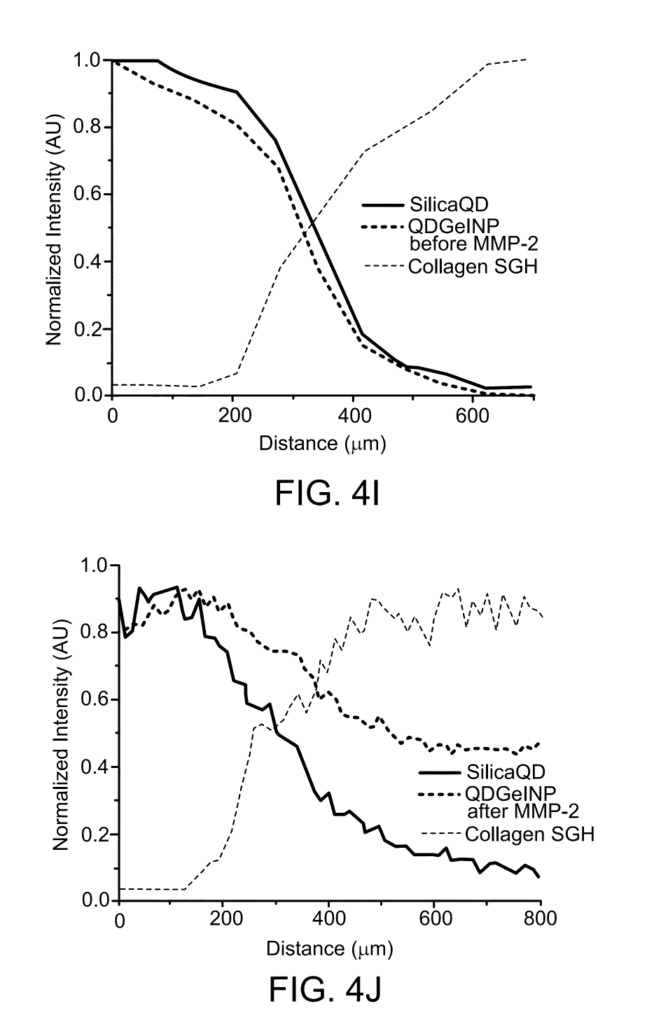

The collagen gel penetration of the QDGelNPs before and after cleaving with MMP-2 was compared to a non-cleavable, PEGylated, and QD-coated silica nanoparticles (Popović Z, et al. (2010), Angewandte Chemie International Edition (accepted)) control (Diam.=105.6±0.8 nm, ζ potential at pH 7.5=−3.9±0.2 mV)—designed to behave like QDGelNPs before cleaving. A mixture of SilicaQDs and QDGelNPs (before or after cleaving) were placed in contact with the gel and incubated for 12 hours. Infiltration of both particles into the collagen was determined using multiphoton microscopy with simultaneous second-harmonic generation (SHG) imaging of fibrillar collagen (FIGS. 4A-H). The SilicaQDs and QDGelNPs before cleaving both had negligible penetration and were excluded from the collagen matrix (FIGS. 4I-J). However, after cleavage of QDGelNPs with MMP-2, the freed QDs were able to penetrate over a millimeter into the gel. By fitting the concentration profile of the cleaved QDGelNPs to a one-dimensional diffusion model (Clauss M A & Jain R K (1990), Cancer Research 50(12):3487-3492; Crank J (1979) The mathematics of diffusion (Oxford University Press, USA)), a diffusion coefficient was obtained of 2.3×10⁻⁷ cm² s⁻¹, the same diffusion coefficient obtained for individual QDs in the collagen gel before conjugation to the gelatin NP. The resulting diffusion coefficient ratio (D/Dₒ, where Dₒ is diffusion coefficient of freed QDs in solution obtained by FCS) in the collagen matrix is 0.49. This value agrees well with the expected value for D/Dₒ of ˜0.52 derived from previous reports (see Methods) (Stylianopoulos T, et al. (2010), Biophysical Journal 99:4; Ramanujan S, et al. (2002), Biophysical Journal 83(3):1650-1660). This result indicates that the diffusion coefficient of released QDs in dense collagen increases to that of ˜10 nm particles and any residual gelatin/glutaraldehyde fragments remaining on the surface do not impede their diffusion.

### Example 4

### MMP-2 Changes Particle Size In Vivo

To test whether tumor secreted MMP-2 can change the size of QDGelNPs in vivo, QDGelNPs and SilicaQDs were co-injected intratumorally in an in vivo tumor model.

Human fibrosarcoma HT-1080 cells were implanted in the dorsal skin of severe combined immunodeficient (SCID) mice for in vivo imaging (Chauhan V P, et al. (2009), Biophysical Journal 97(1):330-336; Leunig M, et al. (1992), Cancer Research 52(23):6553-6560; Jain R K, Munn L L, Fukumura D (2002), Nature Reviews Cancer 2:266-76). When tumors reached 5 mm in diameter, a 1 μL mixture of QDGelNPs and SilicaQDs (about 0.05 μL/min) was injected into the tumor at constant pressure using a glass micropipette connected to a syringe filled with silicone oil.

Images were obtained with a custom-built multiphoton microscope using a Ti:Sapphire laser (Mai-Tai Broadband; Spectra-Physics, Mountain View, Calif.) at 900 nm, a 20×(0.5 NA; Olympus) water-immersion objective, and photon-counting photomultiplier tubes (H7421-40; Hamamatsu). Detection of QDGelNPs was performed via a 530DF100 emission filter and SilicaQDs via a 610DF75 emission filter. Collagen fibers were imaged with second-harmonic generation (16)(33)(44) via a 450DF100 emission filter. The laser power was set to 500 mW. Three dimensional image stacks containing 21 images of 5 μm thickness were obtained wherever fluorescence intensity from the injected particles was detected. A maximum intensity z-projection of each colored stack generated a 2D image. Images of consecutive adjacent regions in the x and y directions were combined into a montage, generating a single image of the entire injection site.

Intensity profiles were extracted using ImageJ and then normalized such that the backgrounds (a “dark” region from all three time-lapse images) had the same intensity. The background was subtracted and the resulting profiles was fitted to the model for diffusing substance initially distributed uniformly through a sphere of radius a (Crank J (1979) The mathematics of diffusion (Oxford University Press, USA)) to obtain the diffusion coefficient:

$$C ( r , t ) = \frac{1}{2} {C}_{o} \{ erf \frac{a - r}{2 \sqrt{Dt}} + erf \frac{a + r}{2 \sqrt{Dt}} \} - \frac{{C}_{o}}{r} \sqrt{\frac{Dt}{π} [ exp \{ - \frac{{( a - r )}^{2}}{4 Dt} \} - exp \{ \frac{{( a + r )}^{2}}{4 Dt} \} ]} ,$$

where Cₒ is the initial concentration in the sphere. It should be noted that the diffusion coefficient obtained in collagen gel was obtained at ˜295° K while the in vivo experiment was measured at the slightly higher body temperature of ˜310° K.

The HT-1080 tumor model was selected because of its reported high MMP-2 activity, which was confirmed by in situ gelatin zymography on a tumor tissue section. The procedure developed in (Mook et al., J. Histochem. Cytochem. 51(6):821-829 (2003)) was used. Cryostat section of an HT-1080 tumor was air-dried for 10 min. DAPI was added to a 1% (w/v) low gelling temperature agarose in PBS to give a final concentration of 1.0 μg/mL. This solution was then combined with a 1.0 mg/mL solution of DQ™ gelatin (Invitrogen, Eugene, Oreg.) in a 10:1 ratio. The combined mixture (40 μl) was put on top of the tumor section and enclosed with a coverslip. After gelation of the agarose at 4° C., the gel and the tumor section was incubated for 2 hours at room temperature. The cell nuclei stained with DAPI and unquenched FITC from MMP digestion of DQ™ gelatin were imaged on a confocal microscope. Fluorescence of FITC was detected with excitation at 460-500 nm and emission at 512-542 nm. DAPI was detected with excitation at 340-380 nm and emission at 425 nm.

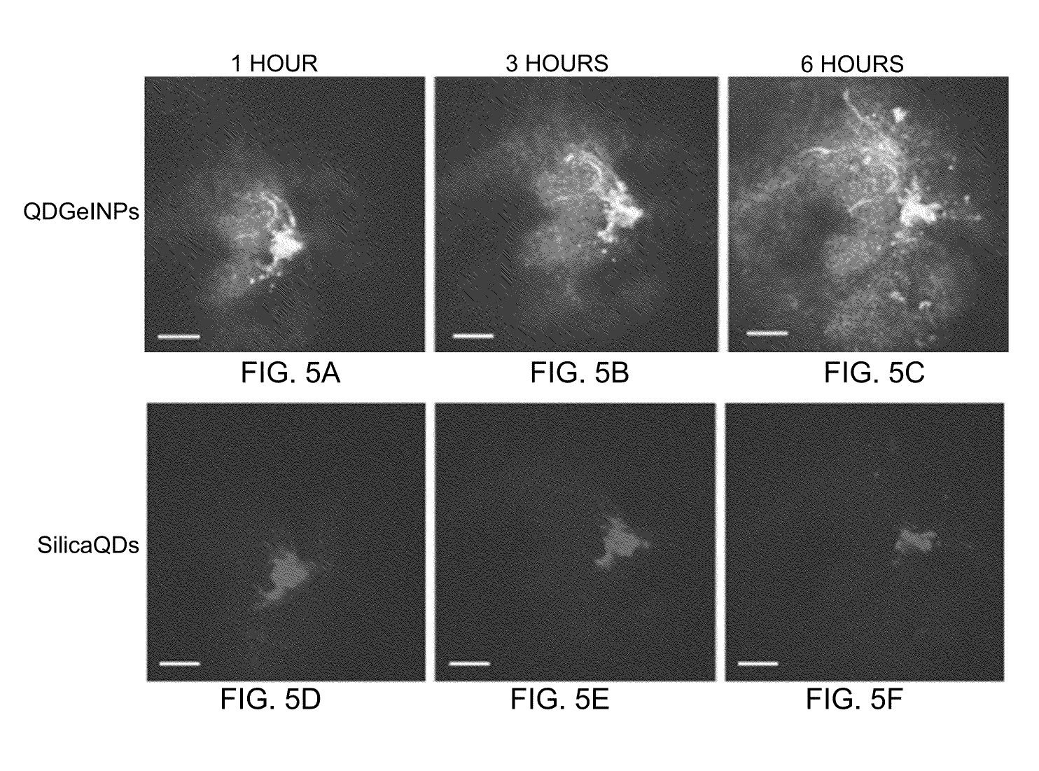

Multiphoton microscopy revealed a marked increase in QDGelNPs penetration into surrounding tumor tissue as compared with the non-cleavable SilicaQDs control, confirming a substantial enhancement in interstitial transport associated with size change (FIGS. 5A-F). At 6 hours post-injection, the QDGelNPs had penetrated up to ˜300 μm from the injection site while the SilicaQDs control exhibited little or no dissemination from its initial location. The concentration profile was fitted to a model for substances diffusing from a spherical source to obtain an effective diffusion coefficient of ˜2.2×10⁻⁸ cm² s⁻¹ inside the tumor. This value is ˜10% the diffusion coefficient obtained in the collagen gel, which can be explained by the increased time needed to cleave the particles, the tortuosity of the interstitial space induced by cellular obstacles (Chauhan V P, et al. (2009), Biophysical Journal 97(1):330-336), and the possibly higher collagen concentration in the FIT-1080 tumor than in the gel.

### Example 5

### Blood Half-Life

Next, the QDGelNPs' blood half-life (t_{1/2β}) was determined to show the QDGelNPs are not rapidly removed from circulation by the reticuloendothelial system (RES). A mixture of the QDGelNPs and SilicaQDs was systemically administered to non-tumor bearing mice by retro-orbital injection and the decrease in fluorescence from both particles was measured in the blood over time. Nanoparticle circulation times were measured in nontumor bearing female SCID mice. Each mouse was anesthetized with a ketamine/xylazine solution before intravenous infusion of nanoparticles by retro-orbital injection. For each time point, starting 1 min before the injection of particles, 13 μL of blood was collected via tail-vein nick and mixed with 3 μL 50 mM EDTA in a plastic 96-well plate. To measure particle concentrations in the blood over time, these samples were imaged using multiphoton microscopy to measure average intensities. Multiphoton imaging was carried out as described previously (Brown E B, Campbell R B, Tsuzuki Y, Xu L, Carmeliet P, Fukumura D, Jain R K (2001), Nature Medicine 7:864-868) on a custom-built multiphoton laser-scanning microscope using confocal laser-scanning microscope body (Olympus 300; Optical Analysis Corp.) and a broadband femtosecond laser source (High Performance MaiTai, Spectra-Physics). Image slices were taken at ˜60 mW at sample surface with depths from 0-200 μm. Mosaic images were taken in raster pattern using a motorized stage (H101, Prior Scientific, Inc.) and customized automation software (LabView, National Instruments). Mosaic stacks were assembled using Image) (NIH, Bethesda, Md.) and Matlab (Math Works). Imaging studies were performed with a 20× magnification, 0.95 NA water immersion objective (Olympus XLUMPlanFl, 1-UB965, Optical Anaylsis). Images were analyzed based on a custom 3D vessel tracing code (Tyrrell et al., (2007) IEEE Trans Med Imaging, 26:223) to create a 3D map of voxel intensity versus distance to the nearest vessel. Images were corrected for sample movement with image registration and autofluorescence intensity correction. To quantify the clearance half-life, a biexponential curve was fit, taking into account tissue absorption and plasma clearance, to the data from each mouse.

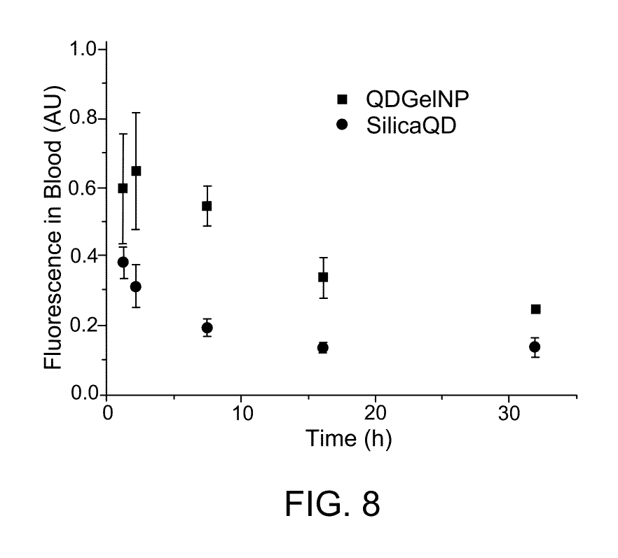

The SilicaQDs exhibited a blood half-life of 12.9±2.4 hours while the QDGelNPs had a half-life of 22.0±3.4 hours (FIG. 8). The difference in the half-lives may be due to variations in the QDGelNPs' surface chemistry that make it less immunogenic compared to SilicaQDs. These results established that QDGelNPs possess both the long circulation half-life and large 100-nm size necessary for preferential extravasation from the leaky regions of the tumor vasculature as well as the deep interstitial penetration of a 10-nm particle required for delivery to the tumor's poorly accessible regions.

These examples demonstrate that a size changing nanostructure can facilitate delivery into the dense collagen matrix of a tumor. The FBS incubation experiment suggested the QDGelNPs are stable in serum conditions but additional studies will be needed to verify that the QDGelNPs are not degraded in the blood by circulating MMPs (Zucker S, Lysik R M, Zarrabi M H, & Moll U (1993) Mr 92,000, Cancer Research 53(1):140-146) and other proteases. Circulating MMPs, however, have been reported to be inhibited by serum proteins such as a2-macroglobulin that entrap the MMP (Chau Y, Tan F E, & Langer R (2004), Bioconjugate Chemistry 15(4):931-941; Woessner J F & Nagase H (2000) Matrix metalloproteinases and TIMPs (Oxford University Press, Oxford, UK)).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a nanostructure comprising gelatin and at least one inner core nanoparticle comprising a cancer therapeutic agent or an imaging agent, wherein the gelatin nanostructure is subject to degradation by MMP-2 and/or MMP-9.

2. The composition of claim 1, wherein the nanoparticle is comprised of one or more members of the group consisting of quantum dots, polymers, liposomes, silicon, silica, dendrimers, microbubbles and nanoshells.

3. The composition of claim 2, wherein the nanoparticle comprises one or more quantum dots.

4. The composition of claim 1, wherein the gelatin nanostructure has a charged outer surface.

5. The composition of claim 4, wherein the outer surface comprises a material selected from the group consisting of polyethylene glycol (PEG), N-(2-hydroxypropyl)methacrylamide (HPMA), poly(vinyl-pyrrolidone) (PVP), poly(ethyleneimine) (PEI), a polyamidoamine, a mixture of divinyl ether and maleic anhydride (DIVEMA (DIVEMA), dextran (alpha-1,6 polyglucose, dextrin (alpha-1,4 polyglucose), hyaluronic acid, a chitosan, a polyamino acid, poly(lysine), poly(glutamic acid), poly(malic acid), poly(sapartamides), poly co-polymers, and copaxone.

6. The composition of claim 1, wherein the nanostructure is about 10-300 nm in diameter.

7. The composition of claim 6, wherein the nanostructure is about 100 nm in diameter.

8. The composition of claim 1, wherein the nanoparticle is about 2-20 nm in diameter.

9. The composition of claim 8, wherein the nanoparticle is about 10 nm in diameter.

10-15. (canceled)
